(19) United States
(12) Patent Application Publication
(10) Pub. No.: US 2024/0050346 A1
(43) Pub. Date: Feb. 15, 2024

(54) NEW COMPOSITION FOR CURLING HAIR

(71) Applicant: DI VISCO, Saint Genis Laval (FR)

(72) Inventors: Sébastien Frédéric VISCOGLIOSI, Charly (FR); Isabel GARCIA, Charly (FR)

(73) Assignee: DI VISCO, Saint Genis Laval (FR)

(21) Appl. No.: 18/268,687

(22) PCT Filed: Dec. 22, 2021
(86) PCT No.: PCT/FR2021/052433

§ 371 Date: Jun. 21, 2023

(30) Foreign Application Priority Data
Dec. 23, 2020 (FR) FR20/14069

(51) Int. Cl. A61K 8/46; A61Q 5/04; A61K 8/44; A61K 8/64; A61K 8/34; A61K 8/92; A61K 8/67; A61K 8/368

(52) U.S. Cl. CPC A61K 8/46; A61Q 5/04; A61K 8/44; A61K 8/64; A61K 8/34; A61K 8/922; A61K 8/676; A61K 8/368; A61K 2800/522

(57) ABSTRACT

A novel composition for curling hair or curling eyelashes includes an amino acid, a protein, an alcohol, a vegetable oil and at least one organic acid; as well as to a method for curling hair or a method for curling eyelashes using said composition.

The present invention relates to a novel composition for curling hair or curling eyelashes comprising an amino acid, a protein, an alcohol, a vegetable oil and at least one organic acid; as well as to a method for curling hair or a method for curling eyelashes using said composition.

In the field of hair care, curling hair corresponds to a hairstyling technique making it possible to curl the hair temporarily or over a longer period.

Consumers wishing to curl their hair are seeking not only to obtain a curly hair with a good hold, but also to obtain a long-lasting result by resisting in particular subsequent shampooing.

To change the shape of the hair in a long-lasting manner, mechanical and/or thermal treatments are generally used.

Mechanical treatments consist in giving wet hair the shape it will keep when dry by winding it over cylindrical tools such as round brushes or curlers while waiting for it to dry.

Heat treatments in turn consist in winding and holding each lock of hair for ten to fifteen seconds on a cylindrical iron heated to a temperature ranging from 160° C. to 230° C.

These treatments allow a visual change of the appearance of the hairstyle, both in terms of shape and in terms of overall volume of the hair. They make it possible to achieve a curly visual appearance, a substantial gain in shine, a resistance to humidity and heat for maintaining the hairstyle all day long.

However, these methods have a very limited effectiveness since the hold of the curls varies from a few hours to a few days only, and the use of tools heated at high temperature causes irreversible damage to the hair.

In order to prolong their effect, the mechanical and thermal treatments are thus generally preceded by “chemical” treatments of the hair by particular compositions.

To obtain the curling of hair, the commonly used technique consists, firstly, in carrying out the opening of disulfide bonds —S—S— of keratin (keratocystin) using a generally basic composition containing a sulfur reducing agent (reduction step) then, after generally rinsing the hair thus treated with water, in secondly reconstituting said disulfide bonds by applying to previously tensioned hair, an oxidizing composition (oxidation step, also called fixing step) so as to finally give the hair the desired shape. The compositions used are generally alkaline or weakly acidic compositions based on thiolated or ammoniated reducing agents. Although the curl effect finally obtained is generally long-lasting, these compositions require strict compliance with the application conditions recommended by the suppliers, in particular in terms of product amount and waiting time. Moreover, they are contraindicated on fragile hair and sensitive scalps due to the highly corrosive effect of these compositions. Problems of broken or even burned hair and irritated scalps are regularly observed. Finally, these compositions are malodorous.

Thus, on the date of the present invention, it remains necessary to develop a composition making it possible to curl the hair in an effective and long-lasting manner, under easy application conditions, without deteriorating the quality of the hair.

The international patent application WO-A-2019/012219 describes a composition allowing an effective and long-lasting hair straightening, according to an application method that is easy to implement while preserving the quality of the hair, said composition comprising an amino acid, a protein, an alcohol and a vegetable oil. The straightening effect is obtained thanks to a process of moderate and reversible denaturation of the fiber which allows it to keep a straight and smooth appearance with a resistance to subsequent shampooing for a period of several months while preserving the integrity of the fiber. This denaturation process is insufficient for use in curling hair.

However, it has now been surprisingly found that a composition similar to that described in the patent application WO-A-2019/012219 to which a certain proportion of one or several organic acid(s) is added made it possible to curl the hair in an effective and long-lasting manner, according to an application method that is easy to implement, while preserving the quality of the hair.

Furthermore, the composition can also be used to curl the eyelashes hair in an effective and long-lasting manner, according to an application method that is easy to implement, while preserving the quality of the eyelashes.

The present invention relates therefore to a composition for curling hair or curling eyelashes comprising:
an amino acid;
a protein;
an alcohol;
a vegetable oil; and
at least 1% (w/w) of at least one organic acid or an organic or mineral salt thereof.

The composition according to the present invention makes it possible to curl the hair in an effective and long-lasting manner, according to an application method that is easy and comfortable to implement while preserving the quality of the hair, in particular due to the absence of thermal tools and thiolated and/or ammoniated reducing agents. Furthermore, the composition can also be used to curl the eyelashes in an effective and long-lasting manner, according to an application method that is easy to implement, while preserving the quality of the eyelashes.

In the context of the present invention:
by “curling the hair” it should be understood any treatment making it possible to curl straight hair in order to give it a spiral or S-shape or to give it an apparent wavy surface;
by “curling the eyelashes” it should be understood any treatment allowing the eyelashes to be rounded in order to give them a more curved shape than their natural shape (that is to say before treatment);
by “amino acid” it should be understood any amino acid known to those skilled in the art, in particular the amino acids of the following general formula (I) or (II):

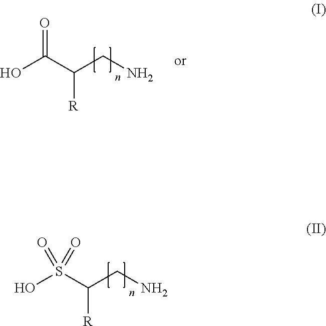

in which
R represents a hydrogen atom, a C₁-C₅-alkyl group optionally
substituted by at least one group selected from hydroxyl, amino; —C(O)—OH; —S(O)₂—OH; —C(O)—O⁻, M+; —S(O)₂—O⁻, M+;
M+ representing a cationic counterion such as alkali metal, alkaline earth metal, or ammonium;
n is 0 or 1;
   as well as betaine forms thereof; optical isomers thereof; solvates thereof such as hydrates; and acid or base, organic or mineral salts thereof;

said amino acids may optionally be grafted onto a heteromolecule possibly being a silicone, a carbohydrate compound, a fatty acid or derivative, a surfactant of anionic, cationic, nonionic or amphoteric nature;
by “vegetable oil” it should be understood any fatty substance extracted by any method of press preparation, centrifugation, esterification or saponification of an oleaginous plant, that is to say a plant whose seeds, nuts or fruits contain lipids, triglycerides or oleic, stearic or linoleic acids, that may optionally be grafted onto a heteromolecule such as a silicone or a surfactant of anionic, cationic, nonionic or amphoteric nature;
by “protein” it should be understood any protein, heteroprotein or protein derivative such as a protein grafted onto a molecule, or a heteromolecule that may be a silicone, a carbohydrate compound, an organic compound, a fatty acid or derivative, a chromophore, a surfactant of anionic, cationic, nonionic or amphoteric nature, of variable size and shape, formed of one or several chain(s) loaded with amino acids and linked by peptide bonds. Preferably, the protein is of tertiary or quaternary structure;
by “Cₓ-C_{y}-alkyl” it should be understood a saturated, linear or branched hydrocarbon chain, and including from x to y carbon atoms;
by “alcohol” “it should be understood any linear or branched carbon chain containing from 1 to 10 carbon atoms substituted by a hydroxyl group, an aminoalcohol, an aldol or a ketol. Preferably, “alcohol” denotes any linear or branched compound of formula CₙH₂ₙ₊₁OH wherein n is an integer comprised between 1 and 10;
by “organic acid” it should be understood any chemical or organic compound, consisting of an acid, polyacid (in particular diacid), hydracid and/or oxacid function due to the presence of a carboxyl (—COOH), hydroxyl (—OH) or enol group, and capable of releasing one or several cations in an aqueous medium, as well as organic or mineral salts thereof; and
by “polyol” it should be understood any C₁-C₁₀-alkyl group substituted by at least two hydroxyl groups. Preferably, by “polyol” it should be understood any compound selected from glycerin, propylene glycol, propane-1,3-diol or sorbitol.

In addition, in the context of the present invention, and unless otherwise stated, the proportions expressed in % correspond to weight percentages relative to the total weight of the relevant entity.

The composition according to the present invention therefore contains an amino acid, a protein, an alcohol, a vegetable oil and at least 1% (w/w) of at least one amino acid. Preferably, the present invention relates to a composition as described above having the following features, considered alone or in combination:
the amino acid is selected from lysine, taurine or a proteinogenic amino acid or an acid or base, organic or mineral salt thereof. Preferably, the amino acid is selected as being a proteinogenic amino acid or an acid or base, organic or mineral salt thereof. More preferably, the amino acid is selected from aspartic acid, glutamic acid, asparagine, carnitine, cysteine, glutamine, histidine, leucine, isoleucine, methionine, N-phenylalanine, proline, hydroxyproline, threonine, tryptophan, tyrosine, valine, glycine, alanine, serine, beta-alanine and arginine or an acid or base, organic or mineral salt thereof. Most preferably, the amino acid is selected from glutamic acid, cysteine and arginine;
the composition contains from 0.1% to 20% of amino acid as defined above, preferably from 0.1% to 5% of amino acid as defined above, more preferably from 0.1% to 3% of amino acid as defined above;
the protein is selected from optionally hydrolyzed creatine, optionally hydrolyzed wheat protein, optionally hydrolyzed silk protein, optionally hydrolyzed spirulina protein, optionally hydrolyzed vegetable keratin, optionally hydrolyzed soy protein, optionally hydrolyzed collagen, optionally hydrolyzed elastin, optionally hydrolyzed pea protein, optionally hydrolyzed rice protein, optionally hydrolyzed conchiolin protein and optionally hydrolyzed whey protein. Most preferably, the protein is selected from creatine, optionally hydrolyzed spirulina protein, optionally hydrolyzed wheat protein, optionally hydrolyzed silk protein and optionally hydrolyzed soy protein;
the composition contains from 0.1% to 20% of protein as defined above, more preferably from 0.1% to 10% of protein as defined above, most preferably from 0.1% to 5% of protein as defined above;
the alcohol is selected from ethanol, butanol, isopropanol and benzyl alcohol. More preferably, the alcohol is selected as being ethanol or benzyl alcohol;
the composition contains from 0.5% to 20% of alcohol as defined above, more preferably from 0.5% to 10% of alcohol as defined above, most preferably from 0.5% to 4% of alcohol as defined above;
the vegetable oil contains at least 1% of linolenic acid or carboxylic acid. More preferably, the vegetable oil is selected from sweet almond oil, soybean oil, argan oil, Haarlem oil, Babassu oil, broccoli oil, avocado oil, linseed oil, apricot oil, cucumber oil, nigella oil, Brazil nut oil, grapeseed oil, shea olein, sesame oil and tomato oil. Most preferably, the vegetable oil is selected as being soybean oil or shea olein;
the composition contains from 0.1% to 10% of vegetable oil as defined above, more preferably from 0.1% to 6% of vegetable oil as defined above, most preferably from 0.1% to 4% of vegetable oil as defined above;
the organic acid is a diacid. Preferably, the organic acid is a diacid with antioxidant properties. More preferably, the organic acid is selected from formic acid, acetic acid, sulphonic acid, carboxylic acid, salicylic acid, acetylsalicylic acid, ascorbic acid, sodium ascorbate, ammonium persulfate, sodium persulfate and potassium persulfate. More preferably, the organic acid is selected from ascorbic acid, salicylic acid and ammonium persulphate. Most preferably, the organic acid is selected as being ascorbic acid;
the composition comprises at least two organic acids. Preferably, the composition comprises at least two organic acids selected as diacids.

More preferably, the composition comprises at least two organic acids selected as diacids with antioxidant properties. More preferably, the composition comprises at least two organic acids selected from formic acid, acetic acid, sulphonic acid, carboxylic acid, salicylic acid, acetylsalicylic acid, ascorbic acid, sodium ascorbate, ammonium persulfate, sodium persulfate and potassium persulfate. More preferably, the composition comprises ascorbic acid and at least one organic acid selected from formic acid, acetic acid, sulphonic acid, carboxylic acid, salicylic acid, acetylsalicylic acid, ammonium persulfate, sodium persulfate and potassium persulfate. Most preferably, the composition comprises ascorbic acid and at least one organic acid selected from salicylic acid, acetylsalicylic acid and ammonium persulfate.
the composition contains from 1% to 20% of organic acid as defined above; more preferably from 1.5% to 15% of organic acid as defined above; most preferably from 3% to 10% of organic acid as defined above; and/or
the composition further contains a polyol.

The composition according to the invention may be in the form of a cream, gel, oil-in-water or water-in-oil emulsion, hair lotion or shampoo. Preferably, the composition according to the invention is in the form of a cream or a hair lotion.

The composition according to the invention may be in the form of a single composition or in the form of a kit comprising two, three or four separate compositions each containing one or several of the ingredients of the composition according to the present invention. Preferably, the present invention also relates to a kit useful for preparing a composition for curling hair as defined above, said kit comprising:
on the one hand a composition containing the protein as defined above and optionally an amino acid, an alcohol, a vegetable oil and/or an organic acid as defined above; And
on the other hand, a composition containing at least an organic acid as defined above and optionally an amino acid, an alcohol and/or a vegetable oil as defined above.

The composition according to the present invention, whether it is in the form of a kit or not, can be applied according to any method known to those skilled in the art.

The composition according to the present invention, when it is used to curl the hair, is preferably applied according to the method comprising the following steps of:
applying the composition according to the invention directly to dry or damp hair;
conventional winding on traditional curlers;
leaving to act for a period that may vary from 5 minutes to 30 minutes;
drying the entire hair while gradually unwinding; and
rinsing the hair with water.

The method according to the present invention can also be supplemented by:
applying a shampoo after rinsing the hair; and/or
applying an after-shampoo balm or a leave-in treatment.

The method according to the present invention can be easily implemented and makes it possible to curl the hair in an effective and long-lasting manner, without the use of a curling iron or thiolated and/or ammoniated reducing agents, thus preserving the quality of the hair.

The composition according to the present invention, when it is used to curl the eyelashes, is preferably applied according to the method comprising the following steps of:
washing the eyelashes with a suitable product;
applying an eyelash “curler” or “pad” type tool on the eyelid;
applying a suitable adhesive in order to hold the eyelashes on the tool;
applying the composition according to the invention to the eyelashes;
leaving to act for a period that may vary from 5 minutes to 30 minutes;
rinsing the eyelashes with a wet cotton ball or a lotion; and
removing the tool.

The method according to the present invention can be easily implemented and makes it possible to curl the eyelashes in an effective and long-lasting manner, without the use of hydrogen peroxide or thiolated and/or ammoniated reducing agents, thus preserving the quality of the eyelashes.

The present invention is illustrated in a non-limiting manner by the following examples.

### EXAMPLE 1—COMPOSITION ACCORDING TO THE INVENTION

A ready-to-use cream comprising an amino acid, a protein, an alcohol, a vegetable oil and two organic acids, the composition of which is reported in the following Table 1, is prepared.

TABLE 1
Curling cream according to the invention

| Ingredients | Amount (% w/w) |
|---|---|
| Arginine (amino acid) | 1.00 |
| Cysteine HCl (amino acid) | 0.60 |
| Hydrolyzed spirulina (protein) | 2.00 |
| Benzyl alcohol (alcohol) | 0.30 |
| Ethanol (alcohol) | 1.50 |
| Soybean oil (vegetable oil) | 2.60 |
| Ascorbic acid (organic acid) | 3.00 |
| Salicylic acid (organic acid) | 1.05 |
| Glycerin | 6.11 |
| Maltose | 3.5 |
| Shea butter | 2.60 |
| Cetearyl alcohol | 2.60 |
| Decyl glucoside | 2.09 |
| Acacia senegal gum | 1.05 |
| Sodium olivoyl glutamate | 0.75 |
| Disodium cocoyl glutamate | 0.75 |
| Xanthan gum | 0.40 |
| Perfume | 0.30 |
| Tocopherol acetate | 0.30 |
| Tocopherol | 0.15 |
| Sunflower oil | 0.15 |
| Sodium chloride | 0.13 |
| Water | 67.07 |

### EXAMPLE 2— HAIR CURLING TEST

2.1—Curling Method

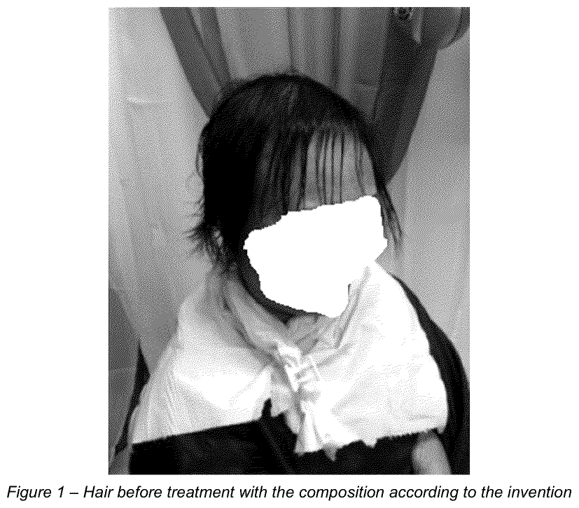

The curling cream described in Example 1 above is used to curl fragile hair, weakened by repeated thiolated perm attempts (FIG. 1).

The application of the composition according to the invention is carried out according to the following protocol.

Step 1: Applying Shampoo Directly to Damp Hair

The composition according to the invention is applied to the entire damp hair and distributed with a comb.

Step 2: Winding on Curlers

The hair thus treated is then wound in a conventional manner on traditional curlers then the product is left for 20 minutes in cellophane and under a climazon hooded hair dryer.

Step 3: Drying and Unwinding

The hair is then dried in hot air with a blow dryer. The curlers are then removed.

Step 4: Rinsing

The hair is then left to cool and rinsed with water, then two successive shampooings are carried out using a shampoo if necessary.

Step 5: Natural Drying

The hair is dried in hot air using a blow dryer or styled with a brush.

2.2—Obtained Results

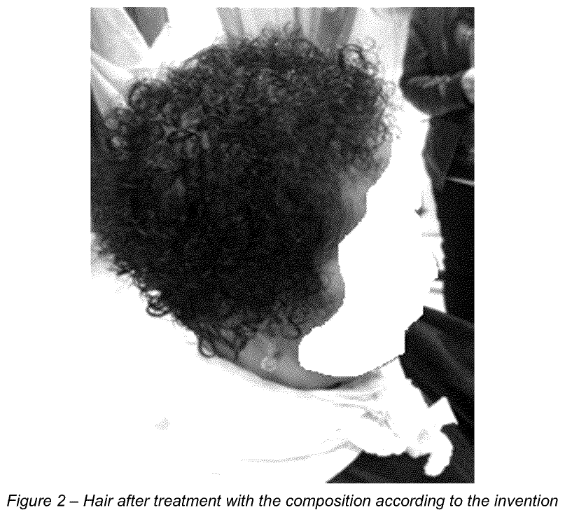

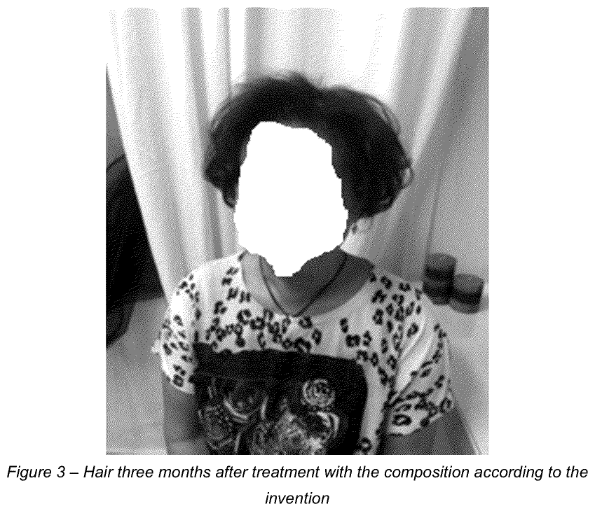

Soft and manageable hair is obtained, with a denser appearance, a significant gain in volume and a significant curl, without any trace of sensitization (FIG. 2). The lifespan observed for this curl is three months (FIG. 3).

### EXAMPLE 3—COMPOSITION ACCORDING TO THE INVENTION

A ready-to-use cream comprising an amino acid, a protein, an alcohol, a vegetable oil and an organic acid, the composition of which is reported in the following Table 2, is prepared.

TABLE 2
Curling cream according to the invention

| Ingredients | Amount (% w/w) |
|---|---|
| Arginine (amino acid) | 1.00 |
| Cysteine HCl (amino acid) | 0.60 |
| Hydrolyzed spirulina (protein) | 2.00 |
| Benzyl alcohol (alcohol) | 0.30 |
| Ethanol (alcohol) | 1.50 |
| Soybean oil (vegetable oil) | 0.10 |
| Ascorbic acid (organic acid) | 3.00 |
| Glycerin | 6.10 |
| Maltose | 3.50 |
| Shea butter | 2.60 |
| Cetearyl alcohol | 2.60 |
| Decyl glucoside | 2.09 |
| Papain | 0.9 |
| Sodium olivoyl glutamate | 0.75 |
| Disodium cocoyl glutamate | 0.75 |
| Xanthan gum | 0.40 |
| Perfume | 0.30 |
| Tocopherol acetate | 0.30 |
| Tocopherol | 0.15 |
| Sunflower oil | 0.15 |
| Sodium chloride | 0.13 |
| Water | 70.78 |

### EXAMPLE 4—HAIR CURLING TEST

4.1—Curling Method

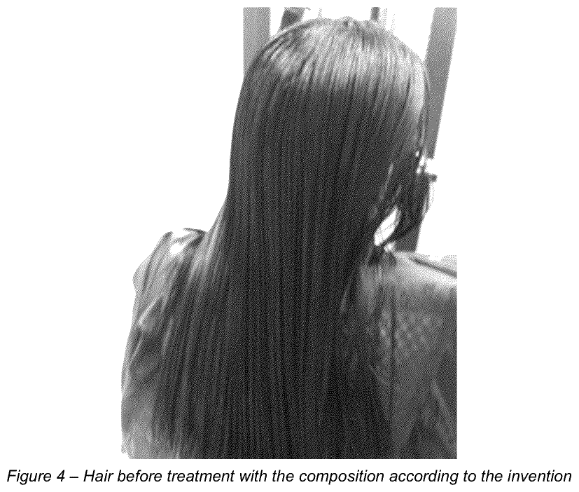

The curling cream described in Example 3 above is used to curl very straight and thick hair, which is usually very difficult to curl and without hold (FIG. 4).

The application of the composition according to the invention is carried out according to the following protocol.

Step 1: Applying a Neutral Shampoo to Clean the Hair.

The composition according to the invention is applied to the entire damp hair and distributed with a comb.

Step 2: Winding on Curlers

The hair thus treated is then wound in a conventional manner on traditional curlers then the product is left for 20 minutes in cellophane and under a climazon hooded hair dryer.

Step 3: Drying and Unwinding

The hair is then dried in hot air with a blow dryer. The curlers are then removed.

Step 4: Rinsing

The hair is then left to cool and rinsed with water, then shampooing is carried out if necessary

Step 5: Natural Drying

The hair is naturally air dried.

4.2—Obtained Results

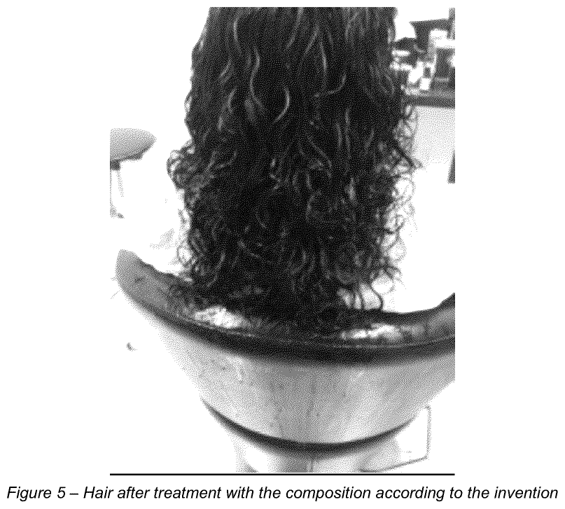

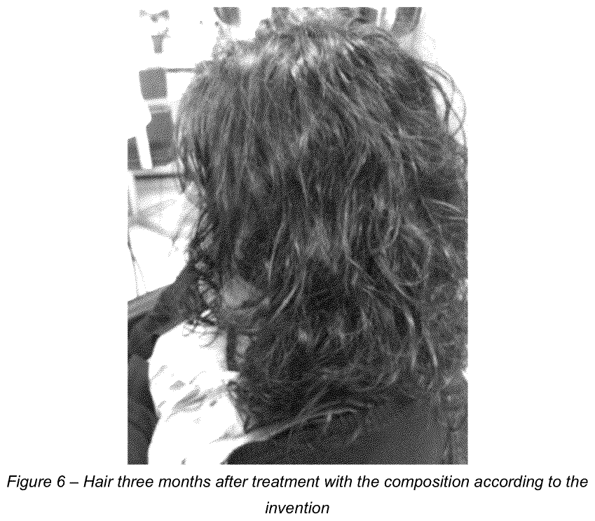

a beautiful bouncy curl, a shiny, soft and strong hair without any sign of dehydration (FIG. 5) are obtained. The observed lifespan of this curl is 3 months (FIG. 6).

### EXAMPLE 5—COMPARATIVE HAIR CURLING TEST

5.1—Tested Composition

A ready-to-use cream comprising an amino acid, a protein, an alcohol and a vegetable oil but containing less than 1% of organic acid is prepared. Its composition is reported in the following Table 3.

This composition corresponds to the straightening composition described in the application WO-A-2019/012219.

TABLE 3
Straightening composition according to WO-A-2019/012219

| Ingredients | Amount (% w/w) |
|---|---|
| Glutamic acid | 0.1 |
| Cysteine HCl | 0.3 |
| Arginine | 0.1 |
| Creatine | 0.05 |
| Hydrolyzed wheat protein | 0.3 |
| Hydrolyzed soy protein | 0.3 |
| Hydrolyzed Keratin | 0.3 |
| Polyquaternium 7 | 0.25 |
| Hydroxypropyl starch phosphate | 0.25 |
| Perfume | 0.3 |
| Phenoxyethanol | 0.3 |
| Amisol Trio ™ | 0.2 |
| Phospholipids | |
| Glycine soybean oil | |
| Glycolipids | |
| Glycine soybean sterol | |
| Ascorbic acid | 0.05 |
| Cetearyl alcohol | 4 |
| Cetrimonium chloride | 2 |
| Ceteareth20 | 0.3 |
| Glycerin | 0.25 |
| Ethylhexylpalmitate | 0.25 |
| Triticum vulgare (wheat germ oil) | 0.25 |
| Orbignya oleifera (Babassu seed oil) | 0.25 |
| Lactic acid | 0.05 |
| Citric acid | 0.05 |
| Ceramide-3 | 0.05 |
| BHT | 0.05 |
| PEG 90 M | 0.1 |
| Water | 89.35 |

5.2—Curling Method

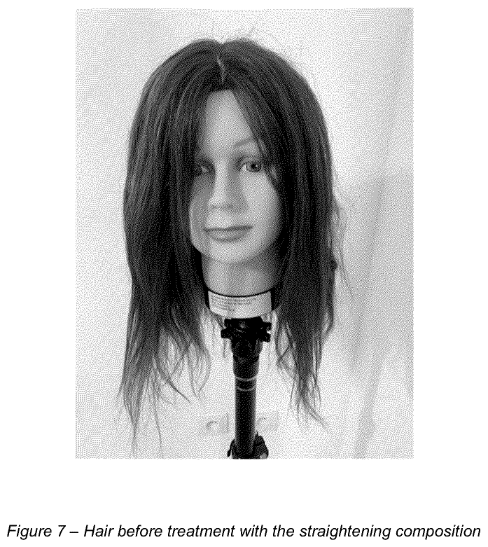

The cream described in the example above is used to check if it is possible to curl straight hair (FIG. 7).

The application of the straightening composition according to the invention is carried out according to the following protocol.

Step 1: Applying Shampoo Directly to Damp Hair

The composition according to the invention is applied to the entire damp hair and distributed with a comb.

Step 2: Winding on Curlers

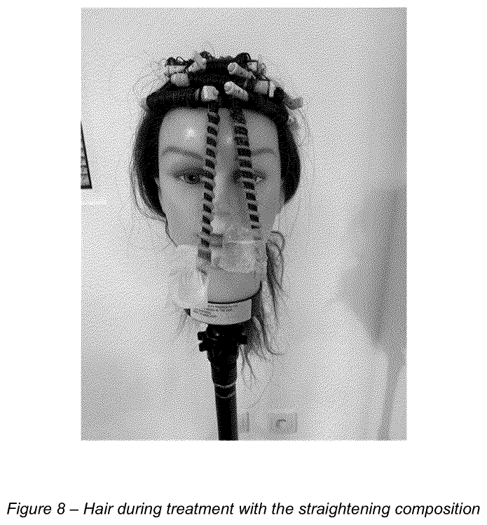

The hair thus treated is then wound in a conventional manner on traditional curlers (FIG. 8) then the product is left for 20 minutes in cellophane and under a climazon hooded air dryer.

Step 3: Drying and Unwinding

The hair is then dried in hot air with a blow dryer. The curlers are then removed.

Step 4: Rinsing

The hair is then left to cool and rinsed with water, then two successive shampooings are carried out using a shampoo if necessary.

Step 5: Natural Drying

The hair is naturally dried at room temperature.

5.3—Obtained Results

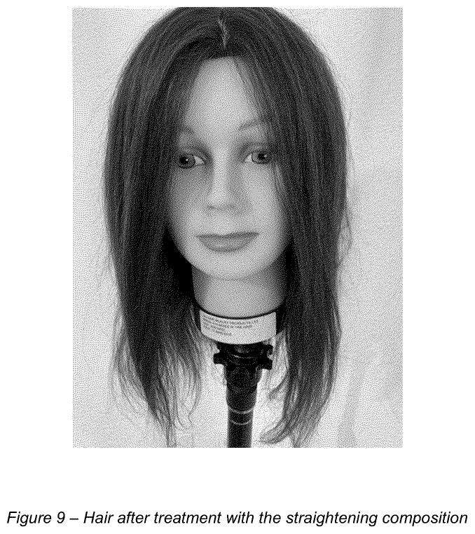

Hair with a denser appearance and a softer fiber is obtained. Nevertheless, no curl is observed (FIG. 9).

The straightening composition described in the application WO-A-20219/012219 therefore does not make it possible to curl the hair to which it is applied.

### EXAMPLE 6—EYELASH CURLING TEST

6.1—Curling Method

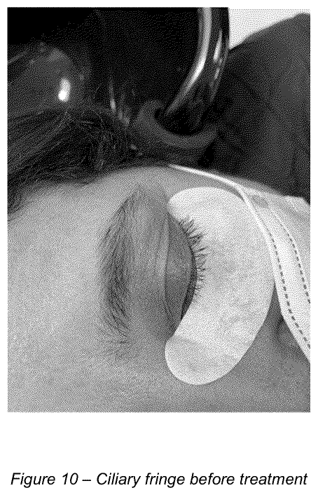

The curling cream described in Example 1 above is used to curl stiff and straight eyelashes (FIG. 10)

The application of the composition according to the invention is carried out according to the following protocol.

Step 1: Cleaning the Eyelashes

The eyelashes are cleaned with a suitable lotion then a curler or an adhesive pad is placed on the upper eyelid

Step 2: Curling the Eyelash

The composition according to the invention is applied to the fringe of the eyelashes using a brush or a blending stump then the product is left for 5 to 30 minutes in cellophane.

Step 3: Rinsing

The product is rinsed with a wet cotton ball then the pad or the curler is peeled off.

6.2—Obtained Results

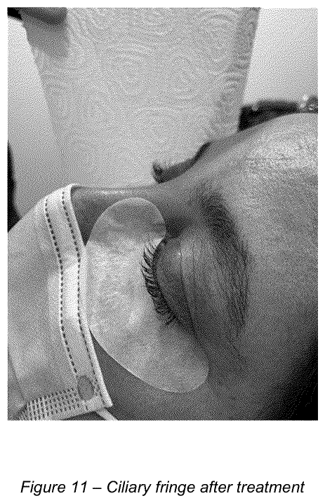

A curved ciliary fringe is obtained with a denser appearance, without any trace of sensitization (FIG. 11). The observed lifespan for this curl is four weeks.

1. A composition for curling hair or curling eyelashes comprising:
an amino acid;
a protein;
an alcohol;
a vegetable oil; and
at least 1% (w/w) of at least one organic acid or an organic or mineral salt thereof.

2. The composition according to claim 1, wherein the amino acid is selected from lysine, taurine or a proteinogenic amino acid or an acid or base, organic or mineral salt thereof.

3. The composition according to claim 2, wherein the amino acid is a proteinogenic amino acid or an acid or base, organic or mineral salt thereof selected from aspartic acid, glutamic acid, asparagine, carnitine, cysteine, glutamine, histidine, leucine, isoleucine, methionine, N-phenylalanine, proline, hydroxyproline, threonine, tryptophan, tyrosine, valine, glycine, alanine, serine, beta-alanine and arginine or an acid or base, organic or mineral salt thereof.

4. The composition according to claim 1, wherein the protein is selected from optionally hydrolyzed creatine, optionally hydrolyzed wheat protein, optionally hydrolyzed silk protein, optionally hydrolyzed spirulina protein, optionally hydrolyzed vegetable keratin, optionally hydrolyzed soy protein, optionally hydrolyzed collagen, optionally hydrolyzed elastin, optionally hydrolyzed pea protein, optionally hydrolyzed rice protein, optionally hydrolyzed conchiolin protein and optionally hydrolyzed whey protein.

5. The composition according to claim 4, wherein the protein is selected from creatine, optionally hydrolyzed spirulina protein, optionally hydrolyzed wheat protein, optionally hydrolyzed silk protein and optionally hydrolyzed soy protein.

6. The composition according to claim 1, wherein the alcohol is selected from ethanol, butanol, isopropanol and benzyl alcohol.

7. The composition according to claim 1, wherein the vegetable oil is selected from sweet almond oil, soybean oil, argan oil, Haarlem oil, Babassu oil, broccoli oil, avocado oil, linseed oil, apricot oil, cucumber oil, nigella oil, Brazil nut oil, grapeseed oil, shea olein, sesame oil and tomato oil.

8. The composition according to claim 7, characterized in wherein the vegetable oil is selected as being soybean oil or shea olein.

9. The composition according to claim 1, wherein the organic acid is a diacid.

10. The composition according to claim 9, wherein the organic acid is a diacid with antioxidant properties.

11. The composition according to claim 10, wherein the organic acid is selected from formic acid, acetic acid, sulphonic acid, carboxylic acid, salicylic acid, acetylsalicylic acid, ascorbic acid, sodium ascorbate, ammonium persulfate, sodium persulfate and potassium persulfate.

12. The composition according to claim 1, wherein it comprises at least two organic acids.

13. The composition according to claim 12, wherein it comprises ascorbic acid and at least one organic acid selected from formic acid, acetic acid, sulphonic acid, carboxylic acid, salicylic acid, acetylsalicylic acid, ammonium persulfate, sodium persulfate and potassium persulfate.

14. A method for curling hair comprising the following steps of:
applying the composition according to claim 1 directly to dry or damp hair;
conventional winding on traditional curlers;
leaving to act for a period that may vary from 5 minutes to 30 minutes;
drying the entire hair while gradually unwinding; and
rinsing the hair with water.

15. A method for curling eyelashes comprising the following steps of:
washing the eyelashes with a suitable product;
applying an eyelash “curler” or “pad” type tool on the eyelid;
applying a suitable adhesive in order to hold the eyelashes on the tool;
applying the composition according to the invention to the eyelashes;
leaving to act for a period that may vary from 5 minutes to 30 minutes;
rinsing the eyelashes with a wet cotton ball or a lotion; and
removing the tool.
